(19) United States
(12) Patent Application Publication
(10) Pub. No.: US 2009/0197949 A1
(43) Pub. Date: Aug. 6, 2009

(54) ANTITUMOR METHODS AND COMPOSITIONS COMPRISING SESQUITERPENE DERIVATIVES

(75) Inventors: Andre PICHETTE, Quebec (CA); Jean Legault, Quebec (CA); Jean-Claude Madelmont, Romagnat (FR)

Correspondence Address: BIRCH STEWART KOLASCH & BIRCH, PO BOX 747, FALLS CHURCH, VA, 22040-0747, US

(73) Assignee: F.P.L. Pharma, Inc., Boucherville (CA)

(21) Appl. No.: 12/422,158

(22) Filed: Apr. 10, 2009

Related U.S. Application Data
(63) Continuation of application No. 10/472,815, filed on May 19, 2004, now abandoned.

(30) Foreign Application Priority Data
Mar. 28, 2001 (CA) 2,342,403

(51) Int. Cl. A61K 31/015; A61K 31/336; A61P 35/00

(52) U.S. Cl. 514/475; 514/766; 514/763

(57) ABSTRACT

The present invention relates to novel antitumor compositions, the use of sesquiterpene derivatives as antitumor agents and to methods of treatment thereof. More precisely, The present invention relates to an antitumor composition comprising a therapeutically effective quantity of at least one sesquiterpene derivative in association with a pharmaceutically acceptable carrier.

### BACKGROUND OF THE INVENTION

(a) Field of the Invention

The present invention relates to novel antitumor compositions, the use of sesquiterpene derivatives as antitumor agents and to methods of treatment thereof.

(b) Description of Prior Art

To date, antitumor activity of α-humulene and isocaryophyllene (γ-caryophyllene) has never been reported in the literature including scientific journals and patents. However, recently Rauter et al. shows a cytotoxic activity of some new humulene derivatives isolated from Asteriscus vogelii (Rauter, A. P. et al.; Phytochemistry, 56, 167-171 (2001). These derivatives include the 8-oxo-α-humula-6Z,9Z-dien-12-oic acid, the 8-oxo-α-humula-6E,9Z-dien-12-oic acid and the 8-oxo-α-humula-6E,9E-dien-12-oic acid. Moreover, an anticarcinogenic activity for β-caryophyllene, β-caryophyllene oxide and α-humulene has been reported by Zheng et al. in 1992 (Zheng, G-Q. et al.; Journal of Natural Products, 55, 999-1003 (1992)). It is important to clarify that anticarcinogenic indicates an inhibition of the carcinogenesis process induced by various chemical agents. The research of Zheng et al. is limited to use these compounds in the prevention of cancers. Indeed, Zheng et al. shows, in vivo, an increasing of gluthatione-S-transferase activity induced by β-caryophyllene, β-caryophyllene oxide and α-humulene. Glutathione-S-transferase is an enzyme responsible of the detoxification of various toxic xenobiotics including the anticarcinogenic agents. Although Zheng et al. suggest that the increasing of glutathione-S-transferase activity could prevent the formation of cancers, they do not demonstrate this hypothesis and do not investigate the antitumoral activity of these compounds.

Antitumoral activity of β-caryophyllene and β-caryophyllene oxide against some solid tumors has been reported by Kubo et al. in Planta Medica (Kubo, I. et al.; Planta Medica, 62, 427-430, (1996)). The antitumoral activity of these two compounds was first evaluated in vitro by the Applicants, but no significant bioactivity for β-caryophyllene and β-caryophyllene oxide was found. Moreover, an other group shows that β-caryophyllene oxide is inactive in vitro against about ten tumor cell lines (Kaneda, N. et al.; Journal of Natural Products, 55, 654-659, (1992)). Finally, Tambe et al., in 1996, demonstrated an gastric cytoprotection effect for β-caryophyllene by inhibition of gastric mucosal injuries induced by necrotizing agents such as absolute ethanol and HCl (Tambe, Y. et al.; Planta Medica. 62, 469-470).

It would be highly desirable to be provided with novel antitumor compositions, the use of sesquiterpene derivatives as antitumor agents and to methods of treatment thereof.

### SUMMARY OF THE INVENTION

One aim of the present invention is to provide novel antitumor compositions, the use of sesquiterpene derivatives as antitumor agents and to methods of treatment thereof.

In accordance with the present invention, there is provided an antitumor composition comprising a therapeutically effective quantity of at least one sesquiterpene derivative in association with a pharmaceutically acceptable carrier.

The sesquiterpene derivative may be extracted from a vegetal oil, including, without limitation, Pinus banksiana oil, Pinus divaricata oil, Pinus strobus oil, Pinus resinosa oil, Pinus sylvestris oil, Picea glauca oil, Picea mariana oil, Thuya occidenitalis oil, Abies balsamea oil and Myrica gale oil.

The sesquiterpene derivative of the present invention includes, without limitation, natural caryophilane, synthetic caryophilane, natural humulane, synthetic humulane and any functional analogs thereof having an antitumor activity.

The preferred humulane is α-humulene.

The preferred caryophilane includes, without limitation, β-Caryophyllene, isocaryophyllene (γ-Caryophyllene), and Caryophyllene oxide.

A preferred antitumor composition of the present invention comprises a therapeutically effective quantity of at least a first sesquiterpene derivative and of a second compound selected from the group consisting of a second sesquiterpene derivative being different than a first derivative, an anti-viral agent, an anti-cancer agent, an immunosuppressive agent, and an anti-inflammatory agent.

Preferably, the antitumor composition of the present invention comprises β-Caryophyllene as a first sesquiterpene derivative and a second sesquiterpene derivative selected from the group consisting of natural caryophilane, synthetic caryophilane, natural humulane, synthetic humulane and any functional analogs thereof.

More preferably, the antitumor composition of the present invention comprises β-Caryophyllene as a first sesquiterpene derivative and a second sesquiterpene derivative being α-humulene or isocaryophyllene.

In accordance with the present invention, there is provided the use of the antitumor composition of the present invention for the preparation of a medicament for reducing tumor growth.

In accordance with the present invention, there is provided the use of the antitumor composition of the present invention for the preparation of a medicament for treatment of a tumor.

In accordance with the present invention, there is provided a method for reducing tumor growth comprising administering a therapeutically effective amount of the antitumor composition of the present invention to a patient.

In accordance with the present invention, there is provided a method for treating a tumor in a patient, said method comprising administering a therapeutically effective amount of the antitumor composition of the present invention to a patient.

For the purpose of the present invention the following terms are defined below.

The term “sesquiterpene derivative” is intended to mean any one of natural caryophilane, synthetic caryophilane, natural humulane, synthetic humulane and any functional analogs thereof having an antitumor activity. Preferably, sesquiterpene derivative includes, without limitation, α-humulene, β-Caryophyllene, isocaryophyllene (γ-Caryophyllene), and Caryophyllene oxide.

The term “humulane” is intended to mean any humulene, without limitation, such as 2,3-epoxy-6,9-humuladiene, 6,7-epoxy-2,9-humuladiene, 9,10-epoxy-2,6-humuladiene, 6,7-epoxy-2-humulen-1-ol, 6,7-epoxy-2-humulene-1,4-diol, 2,6-humuladien-1,4-diol, 2,9-humuladien-7-ol, 2,9-humuladien-6-one, isohumulene, α-humulene, β-Humulene, 3(15),7(14),9-humulatrien-2,6-diol, 1,3(15),6-humulatrien-9-ol, 1,3(15),6-humulatrien-10-ol, 1,3(15),6-humulatrien-14-ol, 2,6,9-humulatrien-14-ol, 2,7(14),9-humulatrien-6-ol, 3(15),6,9-humulatrien-2-ol, 1,3(15),6-humulatrien-8-one, 2,6,9-humulatrien-8-one, humulene bromohydrin, 1-hydroxy-2,6-humuladien-15-al, 1-hydroxy-8-oxo-2,6,9-humulatrien-15oic acid, 15-nor-1,6-humuladien-3-one, 10,13-dihydroxy-humulene, 4,5-epoxy-7-hydroxy-humulene, 1,2-epoxy-13-hydroxy-humulene, 11,13-dihydroxy-1,2-epoxy-humulene, 7-hydroxy-1,2-epoxy-humulene, 7,10-dihydroxy-1,2-epoxy-humulene, 7,10-dihydroxy-1,2-epoxy-humulene, 13-hydroxy-1,2-epoxy-humulene, 11,13-dihydroxy-1,2-epoxy-humulene, 10-hydroxy-1,2-epoxy-humulene, 7,11-dihydroxy-1,2-epoxy-humulene, 7-oxo-1,2-epoxy-humulene, 13-hydroxy-7-oxo-1,2-epoxy-humulene, 10,13-dihydroxy-1,2-epoxy-humulene, 1,2;8,9-diepoxy-humulene, 1,2;8,9-diepoxy-13-hydroxy-humulene, 1,2;8,9-diepoxy-10-hydroxy-humulene, 1,2;8,9-diepoxy-12-hydroxy-humulene, 1,2;4,5-diepoxy-humulene, 1,2;4,5-diepoxy-7-hydroxyhumulene, 1,2;4,5-diepoxy-10-hydroxyhumulene, 4,5;8,9-diepoxy-humulene, 1,2;4,5;8,9-triepoxy-humulene, 2,6-bicyclohumulanedione, and 2-bicyclohumulen-6-one.

The term “caryophyllane” is intended to mean any caryophyllene, without limitation, such as 3(15),6-caryophylladiene, 3(15),6-caryophylladien-5,9-diol, 3(15),6-caryophylladien-14-oic acid, 3,7(14)-caryophylladien-6-ol, 3(15),6-caryophylladien-1-ol, 3(15),6-caryophylladien-4-ol, 3(15),6-caryophylladien-9-ol, 3(15),7-caryophylladien-6-ol, 6-caryophyllen-15-al, 3(15)-caryophyllen-5-one, 12,14-Dihydroxy-3(15),6-caryophylladien-8-one, 3,7-epoxycaryophyllane, 6,7-Epoxy-3(15)-caryophyllene, 6,7-epoxy-3(15)-caryophyllen-12-ol, 9-hydroxy-3(15),6-caryophylladien-14-oic acid, 6-hydroxy-3 (15),7(14)-caryophylladien-8-one, 9-hydroxy-3(15),6-caryophylladien-8-one, 6-hydroxy-6,7-seco-3(15)-caryophyllen-7-one, 6-hydroxy-15-nor-7(14)-caryophyllen-3-one, 6,7-epoxy-15-nor-3-caryophyllanone, Lychnosalicifolide, Naematolin, 14,14′-Oxybis[3(15),6-caryophylladien-8-one], caryophyllen-9β, 10-olide, puliscabrin, punctaporin B, 1β,7β-epoxy-3,5-caryophylladien-15-ol, 4,5-epoxy-caryophyll-8(14)-en-12-ol, 4,5-epoxy-caryophyll-8(14)-en-12-oic acid, 4,5-epoxy-12-nor-caryophyll-8(14)-en-11-ol, 4,5-epoxy-8(14)-caryophyllen-6,12-diol, 4,5-epoxy-8(14)-caryophyllen-7,12-diol, 4,5-epoxy-8(14)-caryophyllen-12,15-diol, 4,5;8(14)diepoxy-caryophyllen-7,12-diol, 4,8-epoxycaryophyllane-5,12,14-triol, 4,5-epoxy-8-oxo-14-norcaryophyllan-12-ol, 4,5-epoxycaryophyllan-12,14-ol, 4,5-epoxy-8(14)-caryophyllen-7-ol, 4,5-epoxy-8-hydroxy-14-caryophyllanal, 3Z,8(14)-caryophylladien-5,12-diol, 5-hydroxy-caryophyll-3Z-en-14al, Caryolandiol, 8-methoxy-5-caryophyllanol, 4-formyl-8-methylen-4,11,11-trimethyl-bicyclo [6.2.0]decane, 4,11-dimethyl-4-formyl-11-hydroxmethyl-8-methylene-bicyclo [6.2.0]decane, 2-acetoxy-9-clovanol, 2-methoxy-9-clovanol, 2,13-epoxy-9-clovanol, 8-formyl-4,11,11-trimethyl-tricyclo[6.3.0.01,9]undecan-5-ol, 14-hydroxycaryophyllene, 14-hydroxycaryophyllen-5,6-epoxide, 14-acetoxy-caryophyllen-5,6-epoxide, caryophyllene-5,6-epoxy-2,12-diol, caryophyllene-5,6-epoxy-2,12-diol monoacetate, 12-acetoxy-7-hydroxy-3(15),6-caryophyllen-8-one, and 7-acetoxy-12-hydroxy-3(1-5),6-caryophyllen-8-one.

The term “tumor” is intended to mean any type of tumor of a cell selected from the group of breast, prostatic, lung, bronchi, gastrointestinal tact, stomach, colon, skin, kidney, brain, leukemic, liver, pancreatic, bone and joint, central nervous system, peripheral nervous system, heart, gonad (ovary), genitourinary, esophageal, pharynx, neuroendocrine, nose and paranasale sinus, and inner ear. The tumor may be benign or malignant. The tumor is intended to include, without limitation, carcinoma, neoplasm, neuroma, carcinoid, blastoma, adenocarcinoma, and melanoma.

The term “pharmaceutically acceptable carrier” is intended to mean any suitable carrier or adjuvant for any mode of administration for providing a mammal, especially a human, with an effective dosage of the antitumor composition of the present invention. For example, mode of administration, includes without limitation, oral, intravenous, intramuscular, subcutaneous, transdermal, parenteral and topical. Dosage forms include, without limitation, tablets, capsules, powders, solutions, dispersions, suspensions, creams, ointments and aerosols.

The term “patient” is intended to mean any mammal patient, which includes, without limitation, human, canine, bovine, porcine, murine, caprine, ovine, feline, and equine.

### BRIEF DESCRIPTION OF THE DRAWINGS

FIG. 1A illustrates the molecular structure of α-humulene;

FIG. 1B illustrates the molecular structure of β-Caryophyllene;

FIG. 1C illustrates the molecular structure of γ-Caryophyllene;

FIG. 1D illustrates the molecular structure of Caryophyllene oxide;

FIG. 2 illustrates the evaluation of antitumor activity of essential oil of pine on murine L1210 tumor cells implanted in B6D2F1 mice; and

FIG. 3 illustrates the evaluation of antitumor activity of β-caryophyllene+humulene compared to β-caryophyllene+isocaryophyllene on murine L1210 tumor cells implanted in B6D2F1 mice.

### DETAILED DESCRIPTION OF THE INVENTION

Surprisingly and in accordance with the present invention, there is provided in vivo evidence supporting the usefulness of the present antitumor composition.

### Cell Culture

Normal human fibroblasts were purchased from Biopredic International (Rennes, France). This frozen culture was obtained from a 36-years old female abdominal surgical waste and the cells used in this work was from the 7^{th} to the 12^{th} passage of the culture. M4Beu and M3DAU, human melanoma cell lines, was established in the laboratory of Dr. J. F. Doré (Institut National de la Santé et de la Recherche Médicale-INSERM, Unit 218, Lyon, France) from metastatic biopsy specimens (Thomasset, N. et al.; British Journal of Cancer, 46, 58-66 (1982)). Murine colon carcinoma cell line CT-26 was obtained from Dr I. J. Fidler (University of Texas M.D. Anderson Cancer Center, Houston). Breast cancer adenocarcinoma MCF-7 and MDA-MB-231, prostatic adenocarcinoma PC-3, ovary adenocarcinoma SK-OV-3, lung carcinoma A-549, colon adenocarcinoma DLD-1, human cell lines and melanoma 1B16 and fibroblast L-929 murine cell lines were purchased from American Type of Culture Collection (ATCC) or the European Collection of Cell Cultures (ECACC; Salisbury, United-Kingdom). Stock cell cultures were maintained as monolayers in 75-cm² culture flasks in Glutamax™ Eagle's minimum essential medium with Earle's salts (Gibco-BRL, Paisley, Scotland) supplemented with 10% foetal calf serum (Sigma), and 5 ml of a 100× solution of vitamins (Gibco), 5 ml of 100 mM sodium pyruvate (Gibco), 5 ml of 100× non-essential amino acids (Gibco) and 2 mg of gentamicin base (Gibco). Cells were grown in a humidified 37° C. incubator containing 5% CO₂.

### In Vitro Survival Assays

Cells were plated at a density of 5×10³ cells per well in 96-well microplates (Nunclon™, Nunc) in 100 μl of culture medium and were allowed to adhere for 16 h before treatment. Then, 100 μl of culture medium containing essential oil of Pinus divaricata, essential oil of Thuya occidentalis, essential oil of Abies balsamea, α-humulene (Sigma-Aldrich); isocaryophyllene (Sigma-Aldrich) were added and incubated at 37° C. for 48 h. All compounds were dissolved in ethanol and the final concentration of ethanol in the culture medium was maintained at 0.5% (v/v). The effect of essential oils and compounds of oil on the proliferation of tumour cells was assessed using resazurin reduction test.

### Resazurin Reduction Test

Resazurin was recently identified as the alamar Blue dye (O'Brien, J. et al.; European Journal of Biochemistry, 267, 5421-6, (2000)). The resazurin reduction test (RRT) was carried out according to the protocol described here. Briefly, plates were rinsed by 200 μl PBS (37° C., Gibco) at 37° C. using an automatic microplate washer (Cell Wash™, Labsystems, Helsinki, Finland) and emptied by overturning on absorbent toweling. Then, 150 μl of a 25 μg/ml solution of resazurin in RPMI 1640 without Phenol red was added in each well using an automatic microvolume dispenser (Multidrop 384™ Labsystems). The plates were incubated 1 h at 37° C. in an humidified atmosphere with 5% of CO₂ for fluorescence development by living cells. Fluorescence was then measured on the automated 96-well plate reader Fluoroskan Ascent FL™ (Labsystems) using an excitation wavelength of 530 nm and an emission one of 590 nm. The fluorescence is proportional to the number of living cells in the well and IC₅₀ (drug concentration required to inhibit cell proliferation by 50%) was calculated from the curve of concentration-dependent survival percentage, defined as the fluorescence in experimental wells as a percentage of that in control wells, with blank values subtracted.

### Evaluation of Antitumor Activity in Mice

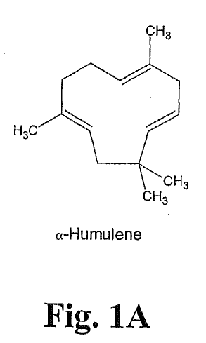

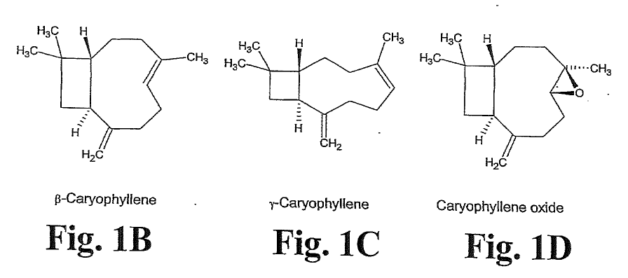

B612F1 male mice, 18-20 g, were obtained from Iffa Credo (Lyon, France). Murine leukemia cell line, L1210 (1×10⁵), were implanted intraperitoneally on day 0. The treatment were administered intraperitoneally on a day -1, -5 and -9 schedule and was delivered as one injection of 0.2 ml/25 g mouse body weight. Each experimental group contained six animals. Each agent including essential pine oil, β-caryophyllene (FIG. 1B), α-humulene (FIG. 1A) and isocaryophyllene (γ-caryophyllene, FIG. 1C) were administered at 1000 mg/kg.

### Identification of Bioactive Natural Products and Results of Biological Activity Studies

The essentials oil of jack pine (Pinus banksiana or Pinus divaricata), eastern white cedar (Thuya occidentalis), balsam fir (Abies balsamea), were evaluated for their cytotoxic activity against various tumor cell lines (Table 1). The cell growth inhibition were measured by the fluorescence induced by the metabolic transformation of resazurin in resorufin. The fluorescence is proportional to living cells and allow to evaluate the concentration inhibiting 50% of cell growth (IC₅₀). Several essential oils have demonstrated a cytotoxic activity against all tumor cell lines tested including: breast adenocarcinoma MCF-7 and MDA-MB-231, prostatic adenocarcinoma PC-3, ovary adenocarcinoma SK-OV-3, lung carcinoma A-549, colon adenocarcinoma DLD-1 and melanoma M4BEU and M3DAU.

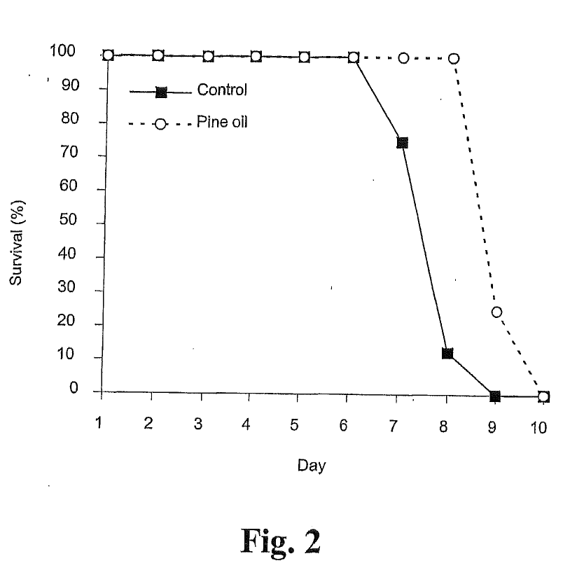

Cytotoxic activity of sesquiterpene oil constituent was evaluated against various tumor cell lines in order to identify bioactive compounds. A sesquiterpene of the humulane family, α-humulene (also known as α-caryophyllene, 2,6,6,9-Tetramethyl-1,4,8-cycloundecatriene) (FIG. 2), is a compound of essential oil demonstrating a significant activity against tumor cells (Table 2). Indeed, α-humulene is active (<100 μmol) against breast adenocarcinoma MCF-7, prostatic adenocarcinoma PC-3, lung carcinoma A-549, colon adenocarcinoma DLD-1 and melanoma M4BEU (Table 2).

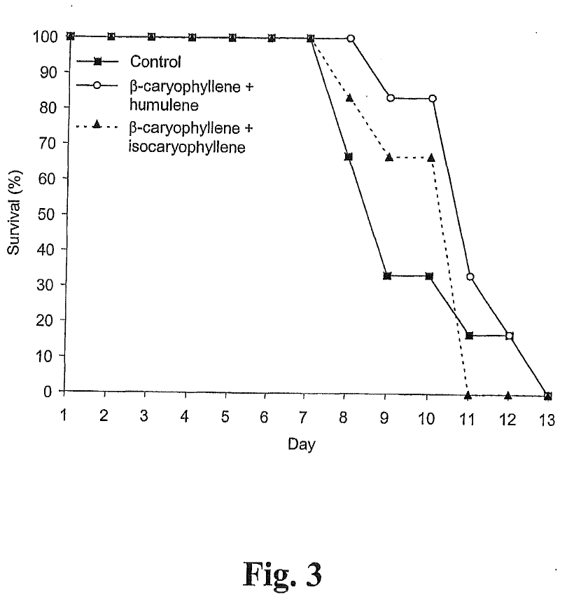

Cytotoxic activity of a sesquiterpene related to α-humulene (FIG. 3), isocaryophyllene, have been evaluated. Isomer cis of β-caryophyllene or isocaryophyllene (also known as γ-caryophyllene, Z-caryophyllene, cis-4,11,11-Trimethyl-8-methylenebicyclo[7.2.0]undec-4-ene) demonstrated as α-humulene, a cytotoxic activity (<100 μmol, Table 2) against all tumor cells tested. Moreover, isocaryophyllene shown to be slightly less active against fibroblastic normal cell in comparison to tumor cells (>100 μmol, Table 2).

Antitumor activity of essential oil of pine on murine L1210 tumor cells implanted in B6D2F1 mice was evaluated. In FIG. 2, the results show that the essential oil of pine increase the survival of mice. Moreover, the antitumor activity of sesquiterpene including β-caryophyllene in combination with humulene and β-caryophyllene in combination with isocaryophyllene was evaluated in vivo. The results appearing in FIG. 3 shows that β-caryophyllene with humulene as well as β-caryophyllene with isocaryophyllene resulted in an increased life span.

TABLE 1
Cytotoxic activity of essential oil of Pinus divaricata, Thuya occidentalis and
Abies balsamea against various tumor cell lines

| | | IC₅₀ (% v,v)^{a} | | |
|---|---|---|---|---|
| | | Pinus | Thuya | Abies |
| Tissue | Cell lines | divaricata | occidentalis | balsamea |
| human breast | MCF-7^{c,d} | 0.11 ± 0.02^{b} | 0.12 ± 0.03^{b} | 0.15 ± 0.07^{b} |
| adenocarcinoma | | | | |
| human breast | MDA-MB-231^{c,d} | 0.067 ± 0.003^{b} | 0.070 ± 0.004^{b} | 0.20 ± 0.01^{b} |
| adenocarcinoma | | | | |
| human prostatic | PC-3^{d,e} | ND^{c} | ND^{c} | 0.19 ± 0.04^{b} |
| adenocarcinoma | | | | |
| human ovary | SK-OV-3^{e} | 0.038 ± 0.002 | 0.043 ± 0.002 | 0.10 ± 0.01^{b} |
| adenocarcinoma | | | | |
| human lung | A-549^{d,e} | 0.028 ± 0.001 | 0.086 ± 0.003 | 0.13 ± 0.03^{b} |
| carcinoma | | | | |
| human colon | DLD-1^{d,e} | ND^{c} | ND^{c} | 0.16 ± 0.03^{b} |
| adenocarcinoma | | | | |
| human melanoma | M4BEU^{f} | 0.047 ± 0.004 | 0.11 ± 0.01 | 0.14 ± 0.03^{b} |
| human melanoma | M3DAU^{f} | 0.064 ± 0.04 | 0.20 ± 0.02 | 0.22 ± 0.07^{b} |
| human fibroblast | Fibroblast^{g} | 0.07 ± 0.02 | 0.16 ± 0.03 | 0.24 ± 0.06^{b} |
| mouse melanoma | B16-F0^{d,e} | ND^{c} | ND^{c} | 0.09 ± 0.02^{b} |
| mouse | L-929^{d,e} | 0.035 ± 0.08 | 0.107 ± 0.008 | 0.18 ± 0.01^{b} |
| subcutaneous | | | | |
| connective tissue | | | | |
| mouse colon | CT-26^{h} | 0.054 ± 0.002 | 0.061 ± 0.004 | 0.087 ± 0.004^{b} |
| carcinoma | | | | |

^{a}Concentration inhibiting cell growth by 50%.
^{b}Values are mean ± SD of three determinations.
^{c}ND: not determined
^{d}ATCC: American Type Culture Collection.
^{e}ECACC (Salisbury, United-Kingdom): European Collection of Cell Culture.
^{f}Thomasset N, Quash G, Dore JF. (1982). Diamine oxidase activity in human melanoma cell lines with different tumorigenicity in nude mice. Br. J. Cancer. Jul; 46(1): 58-66.
^{g}Biopredic International (Rennes, France).
^{h}Brattain MG, Strobel-Stevens J, Fine D, Webb M, Sarrif AM. (1980). Establishment of mouse colonic carcinoma cell lines with different metastatic properties. Cancer Res. Jul; 40(7): 2142-6.

TABLE 2
Cytotoxic activity of sesquiterpenes on various tumor cell lines

IC₅₀ (μM)

| Cells lines | Isocaryophyllene | α-humulene |
|---|---|---|
| MCF-7 | 84 ± 6^{b} | 73 ± 2^{b} |
| PC-3 | 87 ± 8^{b} | 73 ± 2^{b} |
| A-549 | 59 ± 4^{b} | 68 ± 2^{b} |
| DLD-1 | 124 ± 17^{b} | 71 ± 2^{b} |
| M4BEU | 43 ± 3^{b} | 55 ± 2^{b} |
| Fibroblast | 124 ± 15^{b} | 85 ± 5^{b} |
| L-929 | 34.0 ± 0.8^{b} | 50.2 ± 0.3^{b} |
| CT-26 | 46 ± 1^{b} | 53 ± 1^{b} |

^{a}Concentration inhibiting cell growth by 50%.
^{b}Values are mean ± SD of three determinations.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. An antitumor composition comprising an amount of at least one sesquiterpene derivative effective for treating a tumor in association with a pharmaceutically acceptable carrier.

2. The composition of claim 1, wherein said derivative is extracted from an essential oil of a tree.

3. The composition of claim 2, wherein said essential oil is selected from the group consisting of Pinus banksiana oil, Pinus divaricata oil, Pinus strobus oil, Pinus resinosa oil, Pinus sylvestris oil, Picea glauca oil, Picea mariana oil, Thuya occidentales oil, Abies balsamea oil and Myrica gale oil.

4. The composition of claim 1, wherein said derivative is selected from the group consisting of natural caryophilane, synthetic caryophilane, natural humulane, synthetic humulane and any functional analogs thereof.

5. The composition of claim 4, wherein said humulane is α-humulene.

6. The composition of claim 4, wherein said caryophilane is selected from the group consisting of β-caryophyllene, isocaryophyllene, and Caryophyllene oxide.

7. The composition of claim 4, which further comprises a therapeutically effective quantity of at least a second compound selected from the group consisting of a second sesquiterpene derivative, an anti-cancer agent, an immunosuppressive agent, and an anti-inflammatory agent, wherein the second sesquiterpene derivative is different from the first sesquiterpene derivative.

8. The composition of claim 7, wherein said first derivative is β-caryophyllene and said second derivative is selected from the group consisting of natural caryophyllane, synthetic caryophyllane, natural humulane, synthetic humulane and any functional analogs thereof.

9. The composition of claim 8, wherein said second derivative is α-humulene or isocaryophyllene.

10. A method for reducing tumor growth comprising administering a therapeutically effective amount of an antitumor composition comprising a therapeutically effective quantity of at least one sesquiterpene derivative in association with a pharmaceutically acceptable carrier to a patient, wherein the sesquiterpene derivative is selected from the group consisting of α-humulene and isocaryophyllene.

11. The method of claim 10, wherein said tumor is of a cell selected from the group of breast, prostatic, lung, bronchi, gastrointestinal tract, stomach, colon, skin, kidney, brain, leukemic, liver, pancreatic, bone and joint, central nervous system, peripheral nervous system, heart, gonad, genitourinary, esophageal, pharynx, neuroendocrine, nose and paranasale sinus, and inner ear.

12. The method of claim 11, wherein said tumor is selected from the group of carcinoma, neoplasm, neuroma, carcinoid, blastoma, adenocarcinoma, and melanoma.

13. A method for treating a tumor in a patient, said method comprising administering a therapeutically effective amount of an antitumor composition comprising a therapeutically effective quantity of a first sesquiterpene derivative in association with a pharmaceutically acceptable carrier to a patient, wherein the sesquiterpene derivative is selected from the group consisting of α-humulene and isocaryophyllene.

14. The method of claim 13, wherein said tumor is of a cell selected from the group of breast, prostatic, lung, bronchi, gastrointestinal tract, stomach, colon, skin, kidney, brain, leukemic, liver, pancreatic, bone and joint, central nervous system, peripheral nervous system, heart, gonad, genitourinary, esophageal, pharynx, neuroendocrine, nose and paranasale sinus, and inner ear.

15. The method of claim 14, wherein said tumor is selected from the group of carcinoma, neoplasm, neuroma, carcinoid, blastoma, adenocarcinoma, and melanoma.

16-21. (canceled)

22. The method of claim 10, wherein said derivative is extracted from an essential oil of a tree.

23. The method of claim 22, wherein said essential oil is selected from the group consisting of Pinus banksiana oil, Pinus divaricata oil, Pinus strobus oil, Pinus resinosa oil, Pinus sylvestris oil, Picea glauca oil, Picea mariana oil, Thuya occidentalis oil, Abies balsamea oil and Myrica gale oil.

24. The method of claim 10, wherein said sesquiterpene derivative is α-humulene.

25. The method of claim 10, wherein the composition further comprises a therapeutically effective quantity of at least a second compound selected from the group consisting of a second sesquiterpene derivative that is different from the first sesquiterpene derivative and is beta-caryophyllene, an anti-cancer agent, an immunosuppressive agent and an anti-inflammatory agent.

26. The method of claim 13, wherein said derivative is extracted from an essential oil of a tree.

27. The method of claim 26, wherein said essential oil is selected from the group consisting of Pinus banksiana oil, Pinus divaricata oil, Pinus strobus oil, Pinus resinosa oil, Pinus sylvestris oil, Picea glauca oil, Picea mariana oil, Thuya occidentalis oil, Abies balsamea oil and Myrica gale oil.

28. The method of claim 13, wherein said first sesquiterpene derivative is α-humulene.

29. The method of claim 13, wherein said first sesquiterpene derivative is isocaryophyllene.

30. The method of claim 13, wherein the composition further comprises a therapeutically effective quantity of at least a second compound selected from the group consisting of a second sesquiterpene derivative that is different from the first sesquiterpene derivative and is beta-caryophyllene, an anti-cancer agent, an immunosuppressive agent and an anti-inflammatory agent.
